(19) United States
(12) Patent Application Publication
(10) Pub. No.: US 2024/0199513 A1
(43) Pub. Date: Jun. 20, 2024

(54) COMPOSITIONS CONTAINING 3,3,3-TRIFLUOROPROPENE (1243ZF) AND METHODS FOR MAKING AND USING THE COMPOSITIONS

(71) Applicant: THE CHEMOURS COMPANY FC, LLC, WILMINGTON, DE (US)

(72) Inventors: XUEHUI SUN, KENNETT SQUARE, PA (US); MICHAEL A. BRADLEY, HOCKESSIN, DE (US); KARL ROBERT KRAUSE, KENNETT SQUARE, PA (US)

(73) Assignee: THE CHEMOURS COMPANY FC, LLC, WILMINGTON, DE (US)

(21) Appl. No.: 18/287,363

(22) PCT Filed: Apr. 18, 2022
(86) PCT No.: PCT/US2022/025161

§ 371 Date: Oct. 18, 2023

Related U.S. Application Data
(60) Provisional application No. 63/176,602, filed on Apr. 19, 2021.

(51) Int. Cl. C07C 21/18; C07C 19/08; C07C 19/10; C08F 2/48; C08F 4/78; C08F 14/06

(52) U.S. Cl. CPC C07C 21/18; C07C 19/08; C07C 19/10; C08F 2/48; C08F 4/78; C08F 14/06

(57) ABSTRACT

A composition is disclosed that comprises 3,3,3-trifluoropropene (1243zf), 1,1,1-trifluoropropane (263fb), vinyl chloride monomer (CH₂═CHCl), and at least one of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,2,2,2-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

### CROSS REFERENCE TO RELATED APPLICATIONS

This application is a national filing under 35 U.S.C. 371 of International Application No. PCT/US2022/025161 filed Apr. 18, 2022, and claims the benefit of priority of U.S. Provisional Application No. 63/176,602 filed Apr. 19, 2021, the disclosures of which are incorporated herein by reference in its entirety.

### FIELD

The disclosure is directed to compositions containing 3,3,3-trifluoropropene (1243zf) compositions. More particularly, the present disclosure is directed to compositions including 1234zf, 1,1,1-trifluoropropane (263fb), and vinyl chloride monomer (CH₂═CHCl).

### BACKGROUND

Hydrofluorocarbons (HFCs), such as hydrofluoroolefins, have been disclosed as effective refrigerants, fire extinguishants, heat transfer media, propellants, foaming agents, blowing agents, gaseous dielectrics, sterilant carriers, polymerization media, particulate removal fluids, carrier fluids, buffing abrasive agents, displacement drying agents, and power cycle working fluids. Hydrofluoroolefins have replaced chlorofluorocarbons and hydrochlorofluorocarbons, which can potentially damage the Earth's ozone layer. Many hydrofluorocarbons exhibit a high global warming potential (GWP). However, hydrofluoroolefins have a short atmospheric lifespan, due to their reactive olefin bond, and thus do not extensively contribute to global warming.

### SUMMARY

The disclosure relates to compositions comprising 3,3,3-trifluoropropene (1243zf) compositions. The compositions can used for replacing conventional 1243zf as well as a precursor for manufacturing other compounds such as 243db which in turn can be used in processes for manufacturing 1234yf. In one embodiment the inventive 1243zf containing compositions can be used for manufacturing 243db without additional processing or purification and, accordingly, the inventive compositions can provide a cost-effective precursor for making 243db and, if desired, the 243db product can be used in a process for manufacturing 1234yf.

In some embodiments, disclosed herein are compositions including a) 3,3,3-trifluoropropene (1243zf); b), 1,1,1-trifluoropropane (263fb); c) vinyl chloride monomer (VCM/1140/CH₂═CHCl); and d) at least one of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,2,2,2-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

In some embodiments, disclosed herein are compositions contacting a mixture including HF and 1,1,1,3-tetrachloropropane (250fb) in a vapor phase with a fluorination catalyst to effect fluorination of the 250fb. Examples of fluorination catalyst include fluorinated chrome oxide with or without metal dopants. Metal dopants can include zinc, nickel, cobalt, copper, aluminum, among other suitable metal dopants. The fluorination catalyst may be supported, for example, supported on carbon, alumina, silicon carbide, among other supports.

According to any of the foregoing embodiments, also disclosed herein are compositions comprising or consisting essentially of the 1243zf, the 263fb, the VCM, and the at least one compound.

According to any of the foregoing embodiments, also disclosed herein are compositions comprising or consisting of the 1243zf, the 263fb, the VCM, and the at least one compound.

According to any of the foregoing embodiments, also disclosed herein are compositions where the 1243zf is present in the composition in an amount, by mol %, of about 55% or greater.

According to any of the foregoing embodiments, also disclosed herein are compositions where the 263fb is present in the composition in an amount, by mol %, of greater than 0, greater than 0 to about 100 ppm, greater than 0 to about 50 ppm and, in some cases, greater than about 0.0001%.

According to any of the foregoing embodiments, also disclosed herein are compositions where the VCM is present in the composition in an amount, by mol %, of greater than 0, greater than 0 to about 100 ppm, greater than 0 to about 50 ppm and, in some cases, greater than about 0.0001% or greater.

According to any of the foregoing embodiments, also disclosed herein are compositions where the at least one compound is present in the composition in an amount, by mol %, of about 0.00001% or greater.

In some embodiments, disclosed herein are processes including contacting a composition comprising 3,3,3-trifluoropropene (1243zf) with chlorine effecting chlorination of the 3,3,3-trifluoropropene (1243zf) to 2,3-dichloro-1,1,1-trifluoropropane (243db).

According to any of the foregoing embodiments, also disclosed herein are processes where the contacting occurs in the liquid or vapor phase.

According to any of the foregoing embodiments, also disclosed herein are processes where the contacting occurs in the absence of HF.

According to any of the foregoing embodiments, also disclosed herein are processes where the contacting occurs with or without the presence of at least one of chlorination catalyst, and irradiation with an energy source (e.g., UV light). According to any of the foregoing embodiments, also disclosed herein are processes that employ an elevated temperature and, in one particular, embodiment processes that employ an elevated temperature without a catalyst.

According to any of the foregoing embodiments, also disclosed herein are processes where the chlorination catalyst is activated carbon, alumina, chromia, an oxide of another transition metal, a halide of a transition metal, or a combination thereof.

According to any of the foregoing embodiments, the inventive 1243zf compositions can be employed for making fluorosilicone intermediates, fluorosilicone fluids and fluorosilicone rubbers (e.g., as described in U.S. Pat. No. 4,798,818; the disclosure of which is hereby incorporated by reference).

According to any of the foregoing embodiments, the inventive 1243zf compositions can be employed as intermediates for making HFO-1234yf.

The various embodiments can be used alone or in combinations with each other. Other features and advantages of the present invention will be apparent from the following more detailed description, which illustrates, by way of example, the principles of the invention.

### DETAILED DESCRIPTION

The foregoing general description and the following detailed description are exemplary and explanatory only and are not restrictive of the invention.

As used herein, the terms “comprises”, “comprising”, “includes”, “including”, “has”, “having”, or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause, other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements do materially affect the basic and novel characteristic(s) of the claimed invention, especially the mode of action to achieve the desired result of any of the processes of the present invention. The term ‘consisting essentially of’ occupies a middle ground between “comprising” and ‘consisting of’.

Where applicants have defined an invention or a portion thereof with an open-ended term such as “comprising,” it should be readily understood that (unless otherwise stated) the description should be interpreted to also include such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

The term “selectivity”, as used herein, means the ratio of the numbers of moles of the desired product to the number of moles of undesired products expressed as a percentage.

The term “yield”, as used herein, means the ratio of the amount of product produced to the theoretical maximum amount of product, based on the amount of the limiting reagent.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

Provided is a composition including: a) 3,3,3-trifluoropropene (1243zf); b), 1,1,1-trifluoropropane (263fb); c) vinyl chloride monomer (VCM/1140/CH₂═CHCl); and d) at least one of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,2,2,2-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

In exemplary embodiments, the majority of the composition is 3,3,3-trifluoropropene. Appropriate amounts of 3,3,3-trifluoropropene in the composition may include, but are not limited to, by mol %, about 55% or greater, about 70% or greater, about 75% or greater, about 99% or greater, about 99.9% or greater, about 55% to about 95%, about 55% to about 99%, about 55% to about 99.99%, about 60% to about 99%, about 70% to about 99%, about 80% to about 99%, about 90% to about 99%, about 90% to about 99.99%, about 95% to about 99%, about 95% to about 99.99%, about 99.0% to about 99.99%, or any value, range, or sub-range therebetween. If desired, the amount of 3,3,3-trifluoropropene can be greater than 99 and less than 100 mol. %

In exemplary embodiments, the composition also includes 1,1,1-trifluoropropane. Appropriate amounts of 1,1,1-trifluoropropane in the composition may include, but are not limited to, by mol %, greater than 0%, about 0.001% or greater, about 0.01% or greater, about 0.03% or greater, greater than 0% to about 0.001%, about 0.001% to about 1%, about 0.01% to about 1%, about 0.001% to about 0.1%, about 0.01% to about 0.1%, about 0.03% to about 0.1%, about 0.001% to about 0.03%, about 0.03% to about 1%, or any value, range, or sub-range therebetween.

In exemplary embodiments, the composition also includes vinyl chloride monomer. Appropriate amounts of vinyl chloride monomer in the composition may include, but are not limited to, by mol %, greater than 0%, greater than 0% to about 0.001%, about 0.001% or greater, about 0.01% or greater, about 0.001% to about 1%, about 0.01% to about 1%, about 0.001% to about 0.1%, about 0.01% to about 0.1%, or any value, range, or sub-range therebetween.

In exemplary embodiments, the composition also includes at least one of 1,1,1-trifluoroethane, dichlorodifluoromethane, 1-chloro-1-fluoroethylene, 1-chloro-2-fluoroethylene, 1,1-difluoroethane, 1-chloro-2-fluoroethane, 1-chloro-1-fluoroethane, 3-chloro-1,1,1-trifluoropropane, 1,1,1,2-tetrafluoropropane, 1,3,3,3,-tetrafluoropropane, 1,1,1,2-tetrafluoroethane, 1,1,2,2,2-pentafluoropropene, 1,3,3,3-tetrafluoropropene, 3-chloro-3,3,-difluoropropene, dichlorofluoropropene, 1,1,3-trichloropropene, 1,1,1,3-tetrachloropropane, tetrachloroethylene, chloromethane, 2-chloro-3,3,3-trifluoropropene, 1-chloro-3,3,3-trifluoro-1-propene, chlorodifluoropropene, 2-chloro-1,1,1-trifluoropropane, 1,2-dichloro-3,3,3-trifluoropropene, dichlorodifluoropropene, 1,1-dichloroethylene, 2,3-dichloro-1,1,1-trifluoropropane, and pentachlorofluoroethane. In some embodiments, the composition includes at least two, at least three, at least four, at least five, or more than five of the above compounds, Appropriate amounts of the above compounds, alone or in combination, in the composition may include, but are not limited to, by mol %, greater than 0%, 0% to about 0.001%, about 0.001% or greater, about 0.01% or greater, about 0.1% or greater, about 1% or greater, about 0.001% to about 40%, about 0.01% to about 40%, about 0.1% to about 40%, about 1% to about 40%, about 0.001% to about 25%, about 0.01% to about 25%, about 0.1% to about 25%, about 1% to about 25%, or any value, range, or sub-range therebetween.

In exemplary embodiments, the composition is formed by a process of vapor phase fluorination of a composition including 1,1,1,3-tetrachloropropane (250fb) as the primary component.

In exemplary embodiments, the composition is a starting material for the formation of 2,3-dichloro-1,1,1-trifluoropropane (243db).

In some embodiments, the composition is an intermediate in a process of forming 2,3,3,3-tetrafluoropropene (1234yf), which is useful as a low GWP refrigerant, heat transfer medium, and blowing agent.

In some embodiments, the process of forming 2,3,3,3-tetrafluoropropene (1234yf) that includes the composition as an intermediate is a five-step process or a six-step process. In one aspect of this embodiment, the 1243zf compositions of the invention are chlorinated to a 243db containing composition as disclosed in WO2015095497 A1. The resultant 243db containing composition can be dehydrochlorinated to form a 1233xf containing composition as described in WO 2017044724 A1. The resultant 1233xf containing composition can be contacted with HF and a catalyst, for example, 1233xf+HF with SbF5 or SbCl5 catalyst as described in WO 2016187507 A1, in order to produce a 244bb containing composition. In addition, WO 2020018764 A1 discloses converting 243db to 1233xf, 1233xf to 244bb and, if desired, converting 244bb to 1234yf. The disclosure of the previously identified WO publications is hereby incorporated by reference.

In a first reaction, ethylene reacts with carbon tetrachloride to form 1,3,3,3-tetrachloropropane (250fb), as shown in Scheme (1).

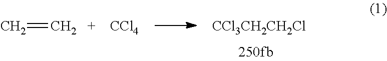

In exemplary embodiments, the reaction of ethylene with carbon tetrachloride occurs as disclosed in International Patent Application No. WO 97/05089, which is incorporated herein by reference.

In some embodiments, the first reaction occurs in the liquid phase. In some embodiments, the first reaction occurs in the vapor phase. In some embodiment, the first reaction occurs in the presence of a catalyst. In some embodiments, the catalyst includes iron, copper, and/or peroxide.

In a second reaction, 1,3,3,3-tetrachloropropane (250fb) undergoes a fluorination reaction to form a composition including 3,3,3-trifluoropropene (1243zf), as shown in Scheme (2).

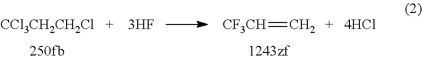

In some embodiments, 250fb is converted to HFC-1243zf by reaction with HF in vapor phase as disclosed in U.S. Pat. No. 6,329,559, which is incorporated herein by reference.

In exemplary embodiments, the fluorination occurs in the vapor phase. The vapor phase fluorination process may be conducted in any reactor suitable for a vapor phase fluorination reaction. The reactor is made of a material that is resistant to the reactants employed. The reactor may be constructed from materials which are resistant to the corrosive effects of hydrogen fluoride such as stainless steel, Hastelloy, Inconel, Monel, gold or gold-lined or quartz. The reactions may be conducted batchwise, continuous, semi-continuous or combinations thereof. Suitable reactors include batch reactor vessels and tubular reactors.

In some embodiments, the vapor phase fluorination includes a fluorination catalyst. In exemplary embodiments, the fluorination catalyst is a chromium on carbon catalyst. Other examples of fluorination catalyst include fluorinated chrome oxide with or without metal dopants. Metal dopants can include zinc, nickel, cobalt, copper, aluminum, among other suitable metal dopants. The fluorination catalyst may be supported, for example, supported on carbon, alumina, silicon carbide, among other supports. In a third reaction, 3,3,3-trifluoropropene (1243zf) undergoes a chlorination reaction to form 2,3-dichloro-1,1,1-trifluoropropane (243db), as shown in Scheme (3).

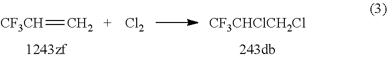

In exemplary embodiments, the starting composition for Scheme (3) is a composition including 1243zf, 263fb, VCM, and at least one of 143a, 12, 1131a, 1131, 152a, 151, 151a, 253fb, 254eb, 254fb, 134a, 1225zc, 1234ze, 1242zf, 1241, 1240za, 250fb, 1110, 40, 1233xf, 1233zd, 1242, 253db, 1223xd, 1232, 1130a, 243db, and 111.

In exemplary embodiments, the chlorination occurs in the vapor phase. In other embodiments, the chlorination occurs in the liquid phase.

In some embodiments, the chlorination includes contacting a starting composition with chlorine in the presence of a catalyst. In some embodiments, the catalyst includes activated carbon, alumina, chromia, and/or an oxide of another transition metal. In another embodiment, the chlorination can include contacting the starting composition with a metal halide (or a metal halide supported such as on carbon, SiC or alumina). In one specific embodiment, the chlorination can include contacting the starting composition with a catalyst comprising FeCl3.

In some embodiments, the chlorination occurs in the absence of HF. In other embodiments, the chlorination occurs in the presence of HF at a mole ratio of HF:1243zf in the range of about 0.01:1 to about 30:1, and about 0.01:1 to about 10:1. In some embodiments, the chlorination occurs at a temperature in the range of about −100 to about 450° C., such as, for example, in the range of about 0 to about 450° C., about 50 to about 350° C. and, in some cases, about 50 to about 250° C.

In some embodiments, the chlorination is a photochemical chlorination. In other embodiments, the chlorination is conducted using elevated temperatures in the absence of a catalyst.

In a fourth reaction, 2,3-dichloro-1,1,1-trifluoropropane (243db) undergoes a dehydrochlorination reaction to form 2-chloro-3,3,3-trifluoropropene (1233xf), as shown in Scheme (4).

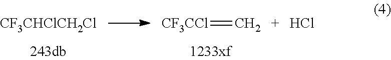

In some embodiments, the dehydrochlorination occurs simultaneously with the chlorination of Scheme (3) in the presence of HF.

In other embodiments, the dehydrochlorination occurs as a separate step from the chlorination of Scheme (3).

In exemplary embodiments, the dehydrochlorination can occur in the liquid or vapor phase. In some embodiments, the dehydrochlorination occurs in the presence of a dehydrochlorination catalyst. Suitable catalysts include activated carbon, alumina, chromium oxide, oxides of transition metals, metal halides, and combinations thereof. The selectivity of the reaction versus the constitutional isomer 1-chloro-3,3,3-trifluoro-1-propene (1233zd) is typically observed over a range of about 1% to about 75%, about 5 to about 60% and, typically, about 20 to about 50%. If hydrogen fluoride (HF) is co-fed with the 243db into the reaction when a Lewis acid type catalyst is used, the formation of 1233zd is suppressed, resulting in improved selectivity of the 1233xf. In some embodiments, the selectivity of 1233xf formation may be at greater than about 92%, greater than about 94%, or greater than about 95%.

In other exemplary embodiments, the dehydrochlorination is performed in the liquid phase by contacting the 243db with a base, such as calcium carbonate, sodium carbonate, potassium carbonate, sodium hydroxide, potassium hydroxide, potassium tert-butoxide, calcium oxides, or calcium hydroxide. The base may be present as a solution in water. The liquid phase dehydrochlorination may be performed in the presence or absence of a phase transfer catalyst. In some embodiments, the phase transfer catalyst includes a quaternary ammonium salt, a phosphonium salt, or a crown ether. The liquid phase process may be performed in the presence or absence of an added solvent such as THE and toluene. The 1233xf product may be separated from the base and the salts formed during the dehydrochlorination reaction by vaporization from the liquid in the reactor.

For a vapor phase dehydrochlorination process, the temperature in the reaction zone may be in the range of about 150 to about 450° C. and about 200° C. to about 400° C. The dehydrochlorination process can be conducted at superatmospheric, atmospheric, or subatmospheric pressures. The contact time of the starting material with the catalyst can be largely varied. Typically, the contact time is from about 2 to about 150 seconds, about 20 to about 120 seconds, and, in some cases, about 10 seconds to about 150 seconds. In some embodiments, the contact time is in the range of about 20 seconds to about 80 seconds.

The contacting step may be carried out by methods known in the art. In some embodiments, starting material, optionally with an inert gas, is fed to a reactor containing the catalyst. In some embodiments of this invention, starting material, optionally with an inert gas, is passed through the catalyst bed in a reactor. In some embodiments of this invention, starting material, optionally with an inert gas, may be mixed with the catalyst in a reactor with stir or agitation.

The dehydrochlorination process may be conducted in the presence of an inert gas such as He, Ar, or N₂. In some embodiments, the inert gas is co-fed into the reactor with the starting material.

In some embodiments, carbons are suitable as the dehydrochlorination catalyst. Carbon used in the embodiments of this invention may come from any of the following sources: wood, peat, coal, coconut shells, bones, lignite, petroleum-based residues and sugar. Commercially available carbons which may be used include those sold under the following trademarks: Barneby & Sutcliffe™, Darco™, Nucharm, Columbia JXN™, Columbia LCK™, Calgon™ PCB, Calgon™ BPL, Westvaco™, Norit™, Takeda™ and Barnaby Cheny NB™.

The carbon also includes three-dimensional matrix porous carbonaceous materials. Appropriate examples are described in U.S. Pat. No. 4,978,649, which is hereby incorporated by reference. In some embodiments, carbon includes three-dimensional matrix carbonaceous materials which are obtained by introducing gaseous or vaporous carbon-containing compounds (e.g., hydrocarbons) into a mass of granules of a carbonaceous material (e.g., carbon black), decomposing the carbon-containing compounds to deposit carbon on the surface of the granules, and treating the resulting material with an activator gas comprising steam to provide a porous carbonaceous material. A carbon-carbon composite material is thus formed.

Embodiments of carbon catalysts include both non-acid washed and acid-washed carbons. In some embodiments, suitable carbon catalysts may be prepared by treating the carbon with acids such as HNO₃, HCl, HF, H₂SO₄, HClO₄, CH₃COOH, and combinations thereof. Acid treatment is typically sufficient to provide carbon that contains less than 1000 ppm of ash. Some suitable acid treatments of carbon are described in U.S. Pat. No. 5,136,113, which is hereby incorporated by reference. In some embodiments, an activated carbon is dried at an elevated temperature and then is soaked for 8 to 24 hours with occasional stirring in 1 to 12 wt % of HNO₃. The soaking process can be conducted at temperatures ranging from room temperature to 80° C. The activated carbon is then filtered and washed with deionized water until the washings have a pH greater than 4.0 or until the pH of the washings does not change. Finally, the activated carbon is dried at an elevated temperature.

In some embodiments, the carbon is an activated carbon. In some embodiments, the carbon is a non-acid washed activated carbon. In some embodiments of this invention, the carbon is an acid washed activated carbon. In some embodiments, the carbon is in the form of powder, granules, or pellets.

The 2-chloro-3,3,3-trifluoropropene (1233xf) may be purified before further use. In some embodiments, the 2-chloro-3,3,3-trifluoropropene (1233xf) is purified by distillation. In one embodiment, the distillation may be performed by heating the reaction mixture to a temperature less than the boiling point of 2,3-dichloro-1,1,1-trifluoropropane (243db) (77° C.) and greater than the boiling point of 2-chloro-3,3,3-trifluoropropene (1233xf), (13° C.). Unreacted 2,3-dichloro-1,1,1-trifluoropropane (243db) may be collected and recycled to the reaction to increase yield.

In a fifth reaction, 2-chloro-3,3,3-trifluoropropene (1233xf) is contacted with hydrogen fluoride, in the presence of a catalyst, and undergoes a hydrofluorination reaction to form 2-chloro-1,1,1,2-tetrafluoropropane (244bb), as shown in Scheme (5).

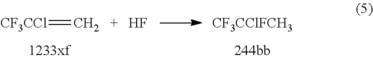

In exemplary embodiments, the hydrofluorination occurs in the liquid phase. In some embodiments, the catalyst is a Lewis acid catalyst such as SbCl₅, TiCl₄, SbF₅, SnCl₄, SbCl₃, TaF₄, or TiF₄. In some embodiments, the Lewis acid catalyst is an antimony-based compound represented by SbClₓF₅₋ₓ. The yield of the reaction is typically in the range of 80% to 99%, or 90% to 99%. The selectivity of the reaction is typically at least 90%. In some embodiments, the selectivity is greater than 95%, greater than 97%, or greater than 99%.

In other embodiments, the hydrofluorination occurs in the vapor phase in the presence of a catalyst. Suitable vapor phase catalysts include antimony chloride on carbon (SbCl₅/C). The selectivity of the vapor phase process may be greater than 95%, greater than 97%, or greater than 98%. Yields have been observed up to about 92%.

In a sixth reaction, 1-chloro-1,1,1,2-tetrafluoropropene (244bb) undergoes a dehydrochlorination reaction to form 2,3,3,3-tetrafluoropropene (1234yf), as shown in Scheme (6).

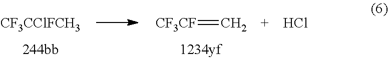

In exemplary embodiments, the dehydrochlorination occurs in the vapor phase. The vapor phase reaction proceeds by thermally dehydrochlorinating the 2-chloro-2,3,3,3-tetrafluoropropane (244bb) to 1234yf, or contacting the 2-chloro-2,3,3,3-tetrafluoropropane (244bb) with a vapor phase dehydrochlorination catalyst to effect dehydrochlorination to form 2,3,3,3-tetrafluoropropene (1234yf). While any suitable catalyst can be employed, an example of a catalyst comprises carbon. Another exemplary embodiment comprises contacting the 2-chloro-2,3,3,3-tetrafluoropropane (244bb) in the liquid phase with a base at a temperature sufficient to effect dehydrochlorination to form 2,3,3,3-tetrafluoropropene (1234yf).

In some embodiments, the dehydrochlorination is conducted in the presence of a dehydrochlorination catalyst. Suitable catalysts include activated carbon, Pd/C, Pt/C, MgF₂, Cr₂O₃, MgO, FeCl₃, CsCl/MgF₂, and KCl/C.

In some embodiments, the dehydrochlorination is conducted without a catalyst by a thermal pyrolysis route. In some embodiments, the reaction mixture is heated to about 460 to 500° C. in the absence of oxygen. Selectivity of greater than 98% may be achieved. It is understood that metal surfaces may have some catalytic effect when the reaction is carried out by the thermal pyrolysis route without a catalyst.

In other embodiments, the dehydrochlorination occurs in the liquid phase by contacting the 244bb with a strong base, such as sodium hydroxide, potassium hydroxide, potassium tert-butoxide, calcium oxides, or calcium hydroxide, in the presence of a catalyst. In some embodiments, the reaction may be performed at a temperature in the range of 70 to 130° C.

In some embodiments, instead of converting 2-chloro-3,3,3-trifluoropropene (1233xf) to 2,3,3,3-tetrafluoropropene (1234yf) by Scheme (5) and then Scheme (6), 2-chloro-3,3,3-trifluoropropene (1233xf) is contacted with hydrogen fluoride, in the presence of a catalyst, and undergoes conversion to form 2,3,3,3-tetrafluoropropene (1234yf), as shown in Scheme (5).

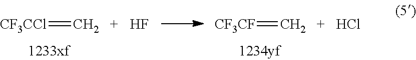

In exemplary embodiments, the conversion occurs in the vapor phase. In some embodiments, the catalyst is a halogenated metal catalyst such as fluorinated chromium oxide, fluorinated Al₂O₃, fluorinated chromium oxide supported on carbon, fluorinated Al₂O₃ supported on carbon, chromium halide, activated carbon, transition metals with activated carbon (e.g., Pt/C).

The products including intermediate products produced by any of the foregoing methods including 2,3,3,3-tetrafluoropropene (1234yf) may be further purified using conventional equipment and methods (e.g., purification using difference in boiling points). Unreacted 1-chloro-1,1,1,2-tetrafluoropropene (244bb) may be recycled to the reaction to increase yield.

While the foregoing inventive methods are effective for making the inventive compositions, the inventive compositions may also be prepared by blending individual components of the inventive compositions.

The following Examples are provided to illustrate certain aspects of the invention and shall not limit the scope of the appended claims.

### EXAMPLES

### Example 1: 250fb Fluorination to 1243zf at 300° C.

A mixture of HF and 250fb at a mol ratio of 20:1 and 0.2 mol % oxygen (O₂) was passed in the vapor phase through a reactor containing fluorinated chromium oxide catalyst at 300° C. A grab sample was taken from the exit of the reactor, washed by a phosphate buffer solution, and then analyzed by GO-MS using an Agilent GO column. The GO analysis products are listed in TABLE 1 below.

TABLE 1

| Compound | Formula | GC-MS area % |
|---|---|---|
| 13 | CClF₃ | 0.0033% |
| 143a | CF₃—CH₃ | 0.0608% |
| 1243zf | CF₃—CH═CH₂ | 58.9078% |
| 12 | CCl₂F₂ | 0.0141% |
| 263fb | CF₃CH₂CH₃ | 0.0256% |
| 254eb | CF₃—CHF—CH₃ | 0.1895% |
| 40 | CH₃Cl | 0.0076% |
| 245fa | CF₃—CH₂—CHF₂ | 0.0588% |
| 3,3,3-trifluoro-2- | CF₃—C(CH₃)═CH₂ | 0.0064% |
| methyl-1-propene | | |
| VCM | CH₂═CHCl | 0.0012% |
| 1234ze | CF₃—CH═CHF | 0.0037% |
| 1233xf | CF₃—CCl═CH₂ | 0.5576% |
| 254fb | CF₃—CH═CHCl | 0.5937% |
| 1242zf | CF₂Cl—CH═CH₂ | 2.7475% |
| 1233zd | CF₃—CH═CHCl | 2.3892% |
| 1348 | | 0.0060% |
| 253db | CF₃—CHCl—CH₃ | 0.9729% |
| 253fb | CF₃—CH₂—CH₂Cl | 26.2613% |
| 1233zd | CF₃—CH═CHCl | 0.2689% |
| 244db | CF₃—CHCl—CH₂F | 0.0359% |
| 1223xd | CF₃—CCl═CHCl | 4.2573% |
| 1232 | C₃H₂F₂Cl₂ | 0.0215% |
| 111 | CCl₃—CFCl₂ | 0.0208% |
| 243db | CF₃—CHCl—CH₂Cl | 0.1904% |
| 1241 isomer | C₃H₃FCl₂ | 0.0814% |
| 1241 isomer | C₃H₃FCl₂ | 0.0429% |
| 1110 | CCl₂═CCl₂ | 0.0318% |
| 1240 | C₃H₃Cl₃ | 0.1348% |
| 250fb | CCl₃—CH₂—CH₂Cl | 0.1778% |
| Others | | 1.9296% |

### Example 2: 250fb Fluorination to 1243zf at 300° C. Followed by Distillation

A mixture of HF and 250fb at a mol ratio of 20:1 and 0.2 mol % oxygen (O₂) was passed in the vapor phase through a reactor containing a fluorinated chromium oxide catalyst at 30000. The product from the exit of the reactor, acids were removed, and the product was then purified by distillation using at a temperature of about 20 to about 25° C. and pressure of about 60 psig, and the distilled product was then analyzed by gas chromatography flame ionization detection (GC-FID) using an Agilent GC column. The GC analysis products are listed in TABLE 2 below.

TABLE 2

| Compound | Formula | GC-FID area % |
|---|---|---|
| 263fb | CF3CH2CH3 | 0.0007 |
| 1131a | CFCl═CH2 | 0.0183 |
| 1131 | CHF═CHCl | 0.0284 |
| 152a | CHF2—CH3 | 0.0034 |
| 1243zf | CF3—CH═CH2 | 86.2845 |
| VCM | CH2═CHCl | 0.0001 |
| HCC-1240za | CH2Cl—CH═CCl2 | 0.0016 |
| HCFC-151 | CH2F—CH2Cl | 0.0284 |
| HCFC-151a | CHFCl—CH3 | 0.0183 |
| HCFC-253fb | CF3—CH2—CH2Cl | 12.2862 |
| HCFC-254eb | CF3—CHF—CH3 | 0.1831 |
| HCFC-254fb | CF3—CH2—CH2F | 0.9214 |
| HFC-134a | CF3—CH2F | 0.0001 |
| HFO-1225zc | CF3—CH═CF2 | 0.001 |
| HFO-1242zf | CF2Cl—CH═CH2 | 0.0471 |
| PCE | CCl2═CCl2 | 0.0106 |
| others | | 0.1668 |

### Example 3: Further Distillation of 1243zf Product

The composition of Example 2 was further purified by distillation at a temperature of about 38 to about 42 C and pressure of about 110 psig and effluent from the distillation column was analyzed by GC-FID using an Agilent column. The GC analysis products are listed in TABLE 3 below.

TABLE 3

| Compound | Formula | GC-FID area % |
|---|---|---|
| 263fb | CF₃CH₂CH₃ | 0.0005 |
| 1131a | CFCl═CH₂ | 0.0001 |
| 1243zf | CF₃—CH═CH₂ | 99.9915 |
| VCM | CH₂═CHCl | 0.0002 |
| HCFC-151a | CHFCl—CH₃ | 0.0001 |
| 152a | CHF₂—CH₃ | 0.0041 |
| HFC-134a | CF₃—CH₂F | 0.0002 |
| HFO-1234ze | CF₃—CH═CHF | 0.0022 |
| others | | 0.0011 |

1243zf was detected as more than 99.99%, and 263fb and VCM were also still detectable in the GC analysis products.

While the invention has been described with reference to one or more embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

1. A composition comprising:
a) 3,3,3-trifluoropropene (1243zf);
b) 1,1,1-trifluoropropane (263fb);
c) vinyl chloride monomer (VCM); and
d) at least one compound selected from the group consisting of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,3,3,3-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

2. The composition of claim 1, wherein the composition consists essentially of the 1243zf, the 263fb, the VCM, and the at least one compound.

3. The composition of claim 1, wherein the composition consists of the 1243zf, the 263fb, the VCM, and the at least one compound.

4. The composition of claim 1, wherein the 1243zf is present in the composition in an amount, by mol %, of about 55% or greater.

5. The composition of claim 1, wherein the 263fb is present in the composition in an amount, by mol %, of about 0.0001% or greater.

6. The composition of claim 1, wherein the VCM is present in the composition in an amount, by mol %, of greater than 0 and less than about 1 wt. %.

7. (canceled)

8. A composition produced by a process comprising:
contacting a mixture comprising HF and 1,1,1,3-tetrachloropropane (250fb) in a vapor phase with a fluorination catalyst to effect fluorination of the 250fb and form the composition, the composition comprising:
a) 3,3,3-trifluoropropene (1243zf);
b) 1,1,1-trifluoropropane (263fb);
c) vinyl chloride monomer (VCM); and
d) at least one compound selected from the group consisting of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,2,2,2-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

9. The composition of claim 8, wherein the fluorination catalyst comprises a chromium on carbon catalyst.

10. The composition of claim 8, wherein the composition consists essentially of the 1243zf, the 263fb, the VCM, and the at least one compound.

11. The composition of claim 8, wherein the composition consists of the 1243zf, the 263fb, the VCM, and the at least one compound.

12. The composition of claim 8, wherein the 1243zf is present in the composition in an amount, by mol %, of about 55% or greater.

13-15. (canceled)

16. A process comprising:
contacting a composition comprising 3,3,3-trifluoropropene (1243zf) with chlorine effect chlorination of the 3,3,3-trifluoropropene (1243zf) to 2,3-dichloro-1,1,1-trifluoropropane (243db);
wherein the composition further comprises 1,1,1-trifluoropropane (263fb); vinyl chloride monomer (VCM); and at least one compound selected from the group consisting of 1,1,1-trifluoroethane (143a), dichlorodifluoromethane (12), 1-chloro-1-fluoroethylene (1131a), 1-chloro-2-fluoroethylene (1131), 1,1-difluoroethane (152a), 1-chloro-2-fluoroethane (151), 1-chloro-1-fluoroethane (151a), 3-chloro-1,1,1-trifluoropropane (253fb), 1,1,1,2-tetrafluoropropane (254eb), 1,3,3,3,-tetrafluoropropane (254fb), 1,1,1,2-tetrafluoroethane (134a), 1,1,2,2,2-pentafluoropropene (1225zc), 1,3,3,3-tetrafluoropropene (1234ze), 3-chloro-3,3,-difluoropropene (1242zf), dichlorofluoropropene (1241), 1,1,3-trichloropropene (1240za), 1,1,1,3-tetrachloropropane (250fb), tetrachloroethylene (1110), chloromethane (40), 2-chloro-3,3,3-trifluoropropene (1233xf), 1-chloro-3,3,3-trifluoro-1-propene (1233zd), chlorodifluoropropene (1242), 2-chloro-1,1,1-trifluoropropane (253db), 1,2-dichloro-3,3,3-trifluoropropene (1223xd), dichlorodifluoropropene (1232), 1,1-dichloroethylene (1130a), 2,3-dichloro-1,1,1-trifluoropropane (243db), and pentachlorofluoroethane (111).

17. The process of claim 16, wherein the contacting occurs in the vapor phase.

18. The process of claim 16, wherein the contacting occurs without a catalyst.

19. The process of claim 16, wherein the contacting occurs in the presence of a chlorination catalyst.

20. The process of claim 19, wherein the chlorination catalyst is selected from the group consisting of activated carbon, alumina, chromia, an oxide of another transition metal, metal halide, and combinations thereof.

21. The process of claim 16, wherein the contacting occurs in the presence of an energy source.

22. The process of claim 21, wherein the source comprises UV light.

23. The process of claim 16, wherein the contacting occurs in the liquid phase.
